(19) United States
(12) Patent Application Publication
(10) Pub. No.: US 2019/0381201 A1
(43) Pub. Date: Dec. 19, 2019

(54) METHOD AND STRUCTURE FOR DIAGNOSIS AND MITIGATING PATHOGENS

(71) Applicant: John Patrick Keady, Fairfax Station, VA (US)

(72) Inventor: John Patrick Keady, Fairfax Station, VA (US)

(21) Appl. No.: 16/244,072

(22) Filed: Jan. 9, 2019

Related U.S. Application Data
(63) Continuation-in-part of application No. 15/922,867, filed on Mar. 15, 2018.
(60) Provisional application No. 62/471,861, filed on Mar. 15, 2017.
(60) Provisional application No. 62/615,152, filed on Jan. 9, 2018.

(51) Int. Cl. A61L 2/025; A61L 2/00; A61N 7/00

(52) U.S. Cl. CPC A61L 2/025; A61L 2/0011; A61N 2007/0073; A61L 2202/24; A61N 7/00

(57) ABSTRACT

At least embodiment is directed to a method of viral eradication which includes: delivering an acoustic wave to a virally infected region; tuning a frequency of the acoustic wave to a resonance frequency of a target virus in the virally infected region; and applying the acoustic wave to the virally infected region for a period of time necessary to eradicate at least 25% of the target virus per cubic mm of the virally infected region.

### CROSS-REFERENCE TO RELATED APPLICATIONS

This application is a continuation in part of U.S. patent application Ser. No. 15/922,867 filed 15 Mar. 2018 the disclosure of which is incorporated in its entirety, and the present application is a non provisional of and claims priority to U.S. Pat. App. No. 62/615,152 filed 9 Jan. 2018 the disclosure of which is incorporated in its entirety. The disclosures of which are all incorporated herein by reference in its entirety.

### FIELD OF THE INVENTION

The present invention relates to devices that can be used to generate of receive acoustical energy and more particularly, though not exclusively, a device that uses acoustical energy to destroy pathogens such as viruses, bacteria and cancer.

### BACKGROUND OF THE INVENTION

Viruses include a genome and often enzymes encapsulated by protein capsid, with often a lipid envelope. A virus must subjugate a host to reproduce, and various methods are used to attack viruses throughout their life cycle. Two common methods used are vaccines and anti-viral drugs. Vaccines can be effective on stable viruses but not on infected patients or fast mutating viruses. Anti-viral drugs target viral proteins. The disadvantage of anti-viral drugs is the eventual pathogen mutation over time and the hazard of side effects if the viral proteins are similar to human proteins.

The market for anti-viral drugs totals in the billions of dollars. Generics in global antivirals market are estimated to be $4.2 billion in 2010 and are forecast to reach $9.2 billion by 2018. Generics in the HIV market accounted for 46% of market share in total generic antivirals market in 2010, while generic herpes therapeutics accounted for 39.6% of market share. Generic influenza therapeutics accounted for 1% of total market share.

It has been reported that in 2002, the annual treatment for HIV/AIDS cost an average of $9,971. This grew substantially at a compound average growth rate (CAGR) of 3.2% to $12,829 in 2010. Deaths in 2011 as a result from HIV/AIDS was greater than 1 million worldwide.

A method of permanent viral eradication, without drugs, without the possibility of pathogen mutation, and with equipment that can treat patients in few visits, would save millions of lives and billions of dollars each year. Additionally the technique could be applied to sterilizing medical instruments.

### BRIEF DESCRIPTION OF THE DRAWINGS

Exemplary embodiments of present invention will become more fully understood from the detailed description and the accompanying drawings, wherein:

FIG. 1 illustrates an acoustic wave impacting a pathogen (e.g., virus); and

FIG. 2 illustrates an acoustic method of diagnosis by impacting the pathogen by an acoustic pulse.

### DETAILED DESCRIPTION OF EMBODIMENTS

The following description of exemplary embodiment(s) is merely illustrative in nature and is in no way intended to limit the invention, its application, or uses.

Exemplary embodiments are directed to a device to generate or receive acoustic waves, that can be used as an acoustic source (e.g., speaker) and acoustic microphone (e.g., microphone). In particular exemplary embodiments discussed utilize fluid-based or laser-based generated acoustic waves to generate high frequency acoustic waves to generate acoustic resonance to deactivate/destroy viruses (MHz to GHz). Note that similar exemplary embodiments can generate hearing acoustic and ultrasonic frequencies (e.g., 10 Hz-50 kHz) and can be used as speakers and microphones.

At least one exemplary embodiment is directed to generating a high frequency acoustic source to set up acoustic resonance in live viruses.

Processes, techniques, apparatus, and materials as known by one of ordinary skill in the art may not be discussed in detail but are intended to be part of the enabling description where appropriate. For example specific materials may not be listed for achieving each of the targeted properties discussed, however one of ordinary skill would be able, without undo experimentation, to determine the materials needed given the enabling disclosure herein. Additionally various techniques, formulas, in acoustical physics and photoacoustics is assumed. Thus the contents of “Photoacoustic Imaging and Spectroscopy” edited by Lihong V. Wang, CRC Press, Optical Scinece and Engineering #144 is incorporated by reference in its entirety, as is the “fundamentals of physical acoustics” by David T. Blackstock, ISBN 0-471-31979-1 which is also incorporated by reference in its entirety.

Notice that similar reference numerals and letters refer to similar items in the following figures, and thus once an item is defined in one figure, it may not be discussed or further defined in the following figures. Processes, techniques, apparatus, and materials as known by one of ordinary skill in the relevant art may not be discussed in detail but are intended to be part of the enabling description where appropriate.

The proposed method utilizes a physical principle well known in the physical sciences called resonance. When an engineering object is designed and built, resonance must be taken into account to avoid catastrophic build up of vibrations that occur at the resonant frequency of the object. The proposed method would gradually build up vibration energy in a virus by impacting the virus with acoustic waves at the virus's resonant frequency, which is a function of the size, density and geometry of the virus. The method, applied for a period of time, would tear apart the targeted virus in a patient's body without interjecting any anti-viral chemicals into the patient's system. The remaining portions of the virus could be used by the immune system of the patient to develop antibodies.

The proposed technique is resonance based and chemical free, and therefore not adaptable by a virus. Resonance is a design concern for any mechanical design. Forced vibrations at the natural frequency of a system can result in resonance buildup to levels that destroy the system, even if the amplitude of the forced vibration is relatively low. The natural frequency of a system depends on several factors, such as size, geometric configuration, density, and damping of the suspension medium.

An acoustical wave impinging upon the object at the resonance frequency will result in a gradual internal amplitude increase to the point in which the object tears itself apart (e.g., exceeds its elastic strength). If a virus is the object, such resonance will be able to tear apart any virus provided the acoustic wave is not detrimentally damped in the medium (e.g. blood) in which the virus lies. The technique can additionally be used for instrument (e.g., medical instrument) sterilization. Different virus's will have different resonant frequencies, and those frequencies will be different than neighboring cells and cellular structure (e.g., size and density differences) such that the virus will be able to be targeted directly without damaging healthy cells.

In general a virus can range in diameter from 20 nm to about 300 nm. If the resonant frequency is solely based upon viral size the needed acoustic frequency would be in the GHz range. The actual viral resonant frequencies are unknown. A simplified air bubble in water model provides a resonant frequency of about 65.6 MHz for a dimension of about 100 nm, much smaller than reported by molecular models.

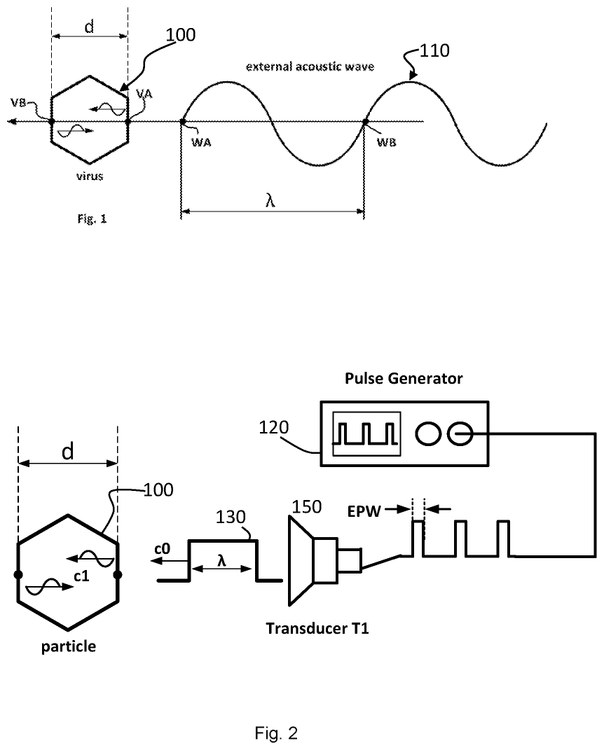

FIG. 1 below provides a simplified view of an acoustic wave 100 passing through a virus 110. Using a simplified assumption (equations (1) and (2) that the speed of sound in the virus is identical to the medium in which it resides, one can see that if the wavelength 140 (λ) of the impinging acoustic wave is twice the viral dimension 170 (d), a new impinging wavelength will strike the virus (e.g., wave part WA 120 impinges virus at location VA 150) when the internal viral acoustic wave 180 reaches the same location (VA 150) after internal reflection (e.g., internal wave reflects from VB 160 back to VA 150 when WB 130 reaches VA 150), allowing resonance buildup. If we assume that the medium is water with a speed of sound of 1500 m/sec, and a viral dimension 170 (d) of 100 nm, we obtain a value of 7.5 GHz. If we reduce the viral dimension to say 10nm the resonance frequency using this simplified model would be 75 GHz. This matches well more complicated molecular models.

d=λ/2 (1)

f=(1500 m/s)/(2*100 nm)=7.5 GHz (2)

A more detailed analysis is provided by equations (3)-(5). Resonance can occur when the time of travel of the internal acoustic wave of the virus from VA->VB->VA matches the period or time of travel of a single wavelength of the ambient acoustic wave. The time of travel (tv) of the internal viral acoustic wave 180 is given in equation (3) and it is a function of the dimension 170 of the virus (d) and the speed of sound in the virus (Cv).

tv=2d/Cv (3)

The period (T) of the ambient acoustic wave 100 is an inverse of the frequency (f) of the wave (in Hz) as expressed by equation (4), and the frequency can be related to the speed of sound in the ambient medium (C0) and the wavelength (λ) of the wave.

T=1/f=λ/C0 (4)

To acquire the condition necessary for resonance to occur, we can set equal T and tv to obtain the expression in equation (5).

T=tv=2d/Cv=λ/C0=1/f (5)

For the simplified case discussed above where Co=Cv, equation (5) reduces to equation (1).

In the simplified view an acoustic source 150 generates an acoustic wave to a virally infected region for a period of time necessary to eradicate at least a target % (e.g., 5%, 10%, 25%, 50%, 75%) of the target virus per cubic mm of the virally infected region. The virally affected region is the region in which the acoustic wave travels that contains the target viruses. For example acoustic source 150 generates the acoustic signal 130 (e.g., 110), which passes through a portion of a pathogen 100. The region through which the acoustic signal passes can include multiple levels (e.g., skin tissue, vascular walls, blood). Each level can affect the acoustic energy (e.g., damping, dispersion) of the acoustic signal. The acoustic signal can be any type of periodic wave (e.g. sine wave, comb functions, ramp functions, cosine waves) that can be used to set up resonance frequencies in the targeted viruses. Since each target virus has a unique size, and composition each virus will have a unique resonance frequency. The acoustic wave 130 can be tuned to the pathogen resonant frequency to eradicate just the target virus. Rupture of the pathogen (e.g., virus, bacteria, cancer cells) can occur although not necessary to render the pathogen sterile and incapable of infecting. A pulse can be used to initiate a pathogen's resonance which can then be picked up by sensors (e.g., ultrasonic transducers) and used to diagnose whether a pathogen exists when the spectral signature (e.g., peaks in FFT) differ from a healthy spectrum. The abnormally appearing frequency peaks can then be used to target the pathogen. The effectiveness of treatment can be monitored by monitoring the reduction of peaks for a given time increment. Additionally an atomic force microscope platform can be modified to oscillate over a frequency range, and when the platform frequency is close to the resonant frequency of the object between the AFM platform and AFM tip the energy transfers more to the AFM which shows up as a peak on the swept AFM diagram. Note that the resonant ultrasonic frequency can be imparted to the pathogen for a period of time necessary to disrupt the pathogen rending it sterile and incapable of continued infection (viral and bacterial) or reproduction (cancer). For example if a viral surface is deformed by greater than 15% from a non deformed state then the virus can be rendered sterile.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures and functions of the relevant exemplary embodiments. For example, if words such as “orthogonal”, “perpendicular” are used the intended meaning is “substantially orthogonal” and “substantially perpendicular” respectively. Additionally although specific numbers may be quoted in the claims, it is intended that a number close to the one stated is also within the intended scope, i.e. any stated number (e.g., 20 mils) should be interpreted to be “about” the value of the stated number (e.g., about 20 mils).

Thus, the description of the invention is merely exemplary in nature and, thus, variations that do not depart from the gist of the invention are intended to be within the scope of the exemplary embodiments of the present invention. Such variations are not to be regarded as a departure from the spirit and scope of the present invention.

What is claimed is:

1. A method of pathogen eradication comprising:
delivering a tuned acoustic signal at the resonant frequency of a pathogen for a period of time necessary to deform the shape of the pathogen by greater than 20%.
